(19) United States
(12) Patent Application Publication
(10) Pub. No.: US 2024/0139427 A1
(43) Pub. Date: May 2, 2024

(54) SURGICAL NEEDLE SAFETY DEVICE AND METHOD

(71) Applicant: Nicholas Mallory, Eagar, AZ (US)

(72) Inventor: Nicholas Mallory, Eagar, AZ (US)

(21) Appl. No.: 18/386,225

(22) Filed: Nov. 1, 2023

Related U.S. Application Data
(60) Provisional application No. 63/421,283, filed on Nov. 1, 2022.

(51) Int. Cl. A61M 5/32

(52) U.S. Cl. CPC A61M 5/3232

(57) ABSTRACT

A surgical needle safety device and method of use is provided. In an embodiment, the safety device is configured for protecting a user and/or patient from injury and/or contamination while handling a surgical needle. The surgical needle safety device includes a needle engagement feature for selective engagement with a needle or other sharp object to fixedly and/or safely retain such needle or other sharp object. The surgical needle safety device comprises a closing mechanism configured for holding the device in a closed configuration with the needle or other sharp object held in a fixed, safe position. In an embodiment, the closing mechanism is spring-loaded to hold the safety device in a closed configuration. In an exemplary embodiment, a user can selectively counteract the spring biasing force to put the device into an open configuration and release the needle or other sharp object.

### CROSS-REFERENCE TO RELATED APPLICATIONS

This application claims priority pursuant to 35 U.S.C. 119(e) to co-pending U.S. Provisional Patent Application Ser. No. 63/421,283, filed Nov. 1, 2022, the entire disclosure of which is incorporated herein by reference.

### FIELD OF THE INVENTION

The present invention relates generally to protective devices and safe handling of sharp and/or contaminated objects. More specifically, the present invention is concerned with a device and method for grasping, retaining, and safely handling surgical needles.

### BACKGROUND OF THE INVENTION

Sutures are commonly used in the medical, dental, and veterinary fields to sew wounds or surgical incisions shut and to promote healing. To close a wound or incision with sutures, a suture needle is attached to the end of a length of suture and threaded through the patient's tissue near the wound or incision at multiple positions, alternating sides of the wound or incision, in a stitched configuration. To effectively perform such suture closures, suture needles must be very sharp to easily puncture through a patient's tissue without unnecessary, additional trauma to the patient's tissues. Moreover, suture needles are typically curved to help the doctor more easily stitch the suture needle and attached suture around the wound or incision.

Accordingly, handling such curved, sharp suture needles can be hazardous to doctors, patients, and/or medical personnel. Traditionally, doctors have held suture needles between the doctor's thumb and forefinger, which can be a major puncture and/or exposure hazard. Other doctors use needle holders while performing suture closure procedures. Nevertheless, even if needle holders are utilized to hold the suture needle, high puncture and/or exposure risk remains when attaching the suture needle to the needle holder; removing the suture needle from the needle holder; and/or cleaning up the surgical station, operating room, etc. Thus, there is need for a device and method for safe handling of a suture needle without puncture or contamination while still allowing a doctor to perform standard suture closure techniques.

Heretofore, there has not been a suture needle handling device or method with the advantages and features of the present invention.

### SUMMARY OF THE INVENTION

The present invention comprises a surgical needle safety device and method for protecting a user and/or patient from injury and/or contamination while handling a surgical needle. In an exemplary embodiment, the present invention includes a needle engagement feature for engaging with a needle or other sharp object and selectively fixedly retaining such needle or other sharp object. In an exemplary embodiment, the present safety device is configured for selectively fixedly retaining at least the sharpened portion of a surgical needle. In exemplary embodiments, the needle safety device is configured for receiving and retaining at least the sharpened portion of a suture needle. Nevertheless, in other embodiments, the device is configured for receiving and retaining a portion or all of another type of surgical needle, a sewing needle, any other type of needle, or another sharp or contaminated object.

In an exemplary embodiment, the surgical needle safety device comprises a closing mechanism configured for holding the device in a closed configuration with the needle or other sharp object held in a fixed, safe position. In an exemplary embodiment, the closing mechanism utilizes a biasing device configured to hold the safety device in a closed configuration. In an exemplary embodiment, a user can selectively counteract the biasing force to put the device into an open configuration and release the needle or other sharp object.

In an exemplary embodiment of the present invention, the suture needle safety device comprises a hinged proximal end opposite a distal, grasping end. In this embodiment, the device further comprises first and second arms connected to each other via the proximal, hinged end. The distal ends of the first and second arms form the safety device distal, grasping end. In an exemplary embodiment, the proximal, hinged end includes an internal torsion spring configured to bias the first and second arms in an open configuration. In other embodiments, the hinged end includes an alternative biasing feature to bias the device into the open configuration. In an exemplary embodiment, a user can apply pressure to each of the first and second arms to counter the hinged end biasing force and close the first and second arms toward each other. In an exemplary embodiment, each of the first and second arms has a slightly concave shape inward, or toward the other arm. In other embodiments, the first and second arms are substantially straight.

In an exemplary embodiment, the suture needle safety device comprises a needle engagement feature positioned near the distal, grasping end. In an exemplary embodiment, the needle engagement feature comprises a paddle or substantially oval-shaped needle engagement surface mounted on each of the first and second arms of the needle safety device. In an exemplary embodiment, these engagement paddles are configured to align and to closely fit together and around a suture needle or other sharp object in a fixed position when the device is in a closed configuration. In an exemplary embodiment, the paddles each include a knurled surface for increased gripping ability. In other embodiments, the paddles include alternative gripping surfaces.

In an exemplary embodiment, the suture needle safety device further includes a closing mechanism comprising an extension post, extension post receiver, and a biased latch configured to engage with the extension post. In an exemplary embodiment, one arm of the suture needle safety device mounts the extension post and the other arm defines the extension post receiver with a corresponding spring-loaded latch. In an embodiment, the extension post comprises a post head which extends outward generally proximally and distally from the rest of the extension post. In an embodiment, the post head is angled with a lower side facing the post receiver latch opposite a post head higher side. Such shape facilitates easy engagement of the extension post in and through the extension post receiver when the device is closed by retracting the latch until the post head extends through the extension post receiver. At such point, the spring-loaded latch expands and contacts the extension post below the post head. This engagement of the extension post, extension post receiver, and corresponding latch holds or locks the device in a closed configuration until the closing mechanism latch is retracted and the extension post disengaged from the extension post receiver, allowing the hinge end biasing feature to bias the device into the open configuration.

In an exemplary embodiment, the suture needle safety device distal end further comprises narrowed graspers or pinchers positioned distal of the needle engagement feature. Such narrowed graspers allow a user to use the device to grasp other objects, such as but not limited to other surgical procedure materials or tissue surrounding an incision to be sutured shut. In an embodiment, the narrowed graspers are grooved to aid in grasping objects or materials. In other embodiments, the graspers include alternative gripping material.

In an embodiment, the device further includes a guide piece mounted from one arm and a corresponding guide receiver positioned on the other arm. Such guide piece and receiver are configured to guide closure of the device arms into a closed configuration and to ensure proper alignment of the needle engagement feature and/or graspers.

In an exemplary embodiment, the suture needle safety device further comprises a finger engagement feature positioned near the device proximal end. In an embodiment, the finger engagement feature comprises a finger loop. In an exemplary embodiment, the finger engagement feature is adjustable for comfortable fit for a user's hand, such as but not limited to adjustment by ratcheted connection.

In an alternative embodiment of the present invention, a suture needle safety device comprises a main body having a proximal end opposite a distal end. In this embodiment, the device comprises a needle engagement feature at the main body distal end. The main body mounts a spring-loaded slider mechanism configured to engage with a series of generally beak-shaped pivotable pieces at the main body distal end. The slider mechanism spring biases the slider distally, which causes the pivotable pieces to pivot toward each other to form a closed needle engagement feature in a device closed configuration. When a user pulls or slides back the slider, counteracting the biasing force of the spring, the pivotable pieces pivot to open a needle receiver cavity in an open configuration for housing at least a sharp end of a suture needle. With a needle positioned within the needle receiver cavity, release of the slide puts the device in a closed configuration with the needle fixedly retained between the pivotable pieces. In an embodiment, the main body comprises a finger engagement feature and/or optional gripping surface to aid a user in handling the suture needle safety device.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

Embodiments of the present invention are described in detail below with reference to the attached drawing figures, wherein:

FIG. 1 is an upper, proximal, perspective view of a suture needle safety device embodying the present invention, with the device in an open configuration.

FIG. 2 is a lower, perspective view of the suture needle safety device in the open configuration.

FIG. 3 is a side, elevational view of the suture needle safety device in the open configuration.

FIG. 4 is a side, elevational view of the suture needle safety device in a closed configuration.

FIG. 5 is a top, plan view of the suture needle safety device.

FIG. 6 is a bottom, plan view of the suture needle safety device.

FIG. 7 is a distal end, elevational view of the suture needle safety device.

FIG. 8 is a proximal end, elevational view of the suture needle safety device.

FIG. 9 is an upper, perspective view of an alternative embodiment of a suture needle safety device of the present invention, with the device in a closed configuration around a suture needle.

FIG. 10 is a side, elevational view of the alternative suture needle safety device in the closed configuration.

FIG. 11 is a side, elevational view of the alternative suture needle safety device in an open configuration.

FIG. 12 is a top, plan view of the alternative suture needle safety device.

FIG. 13 is a distal end, elevational view of the alternative suture needle safety device.

FIG. 14 is a proximal end, elevational view of the alternative suture needle safety device.

The drawing figures do not limit the present invention to the specific embodiments disclosed and described herein. The drawings are not necessarily to scale, emphasis instead being placed upon illustrating the principles of the invention.

### DETAILED DESCRIPTION OF THE PREFERRED EMBODIMENTS

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The present invention comprises a safety device and method of use thereof. In an exemplary embodiment, the present safety device is configured for protecting a user and/or patient from injury and/or contamination while handling a surgical needle. In an exemplary embodiment, the present invention includes a needle engagement feature for selective engagement with a needle or other sharp object to fixedly and/or safely retain such needle or other sharp object. In an exemplary embodiment, the present safety device is configured for selectively fixedly retaining at least the sharpened portion of a surgical needle. In exemplary embodiments, the needle safety device is configured for receiving and retaining at least the sharpened portion of a suture needle. In other embodiments, the device is configured for receiving and retaining a portion or all of another type of surgical needle, a sewing needle, any other type of needle, another sharp object, a contaminated object, or any other object desired to be safely and/or fixedly retained.

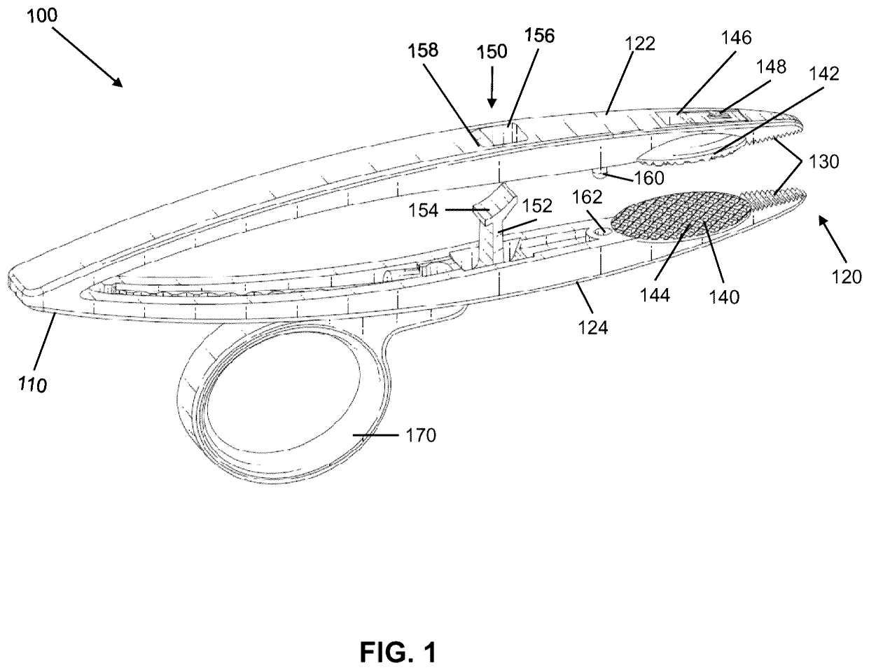

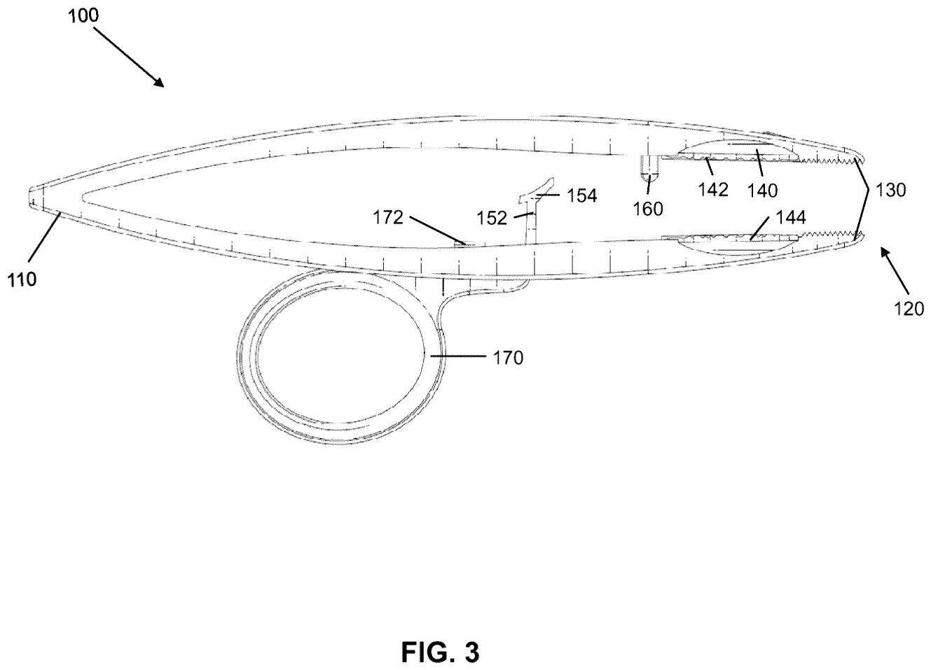

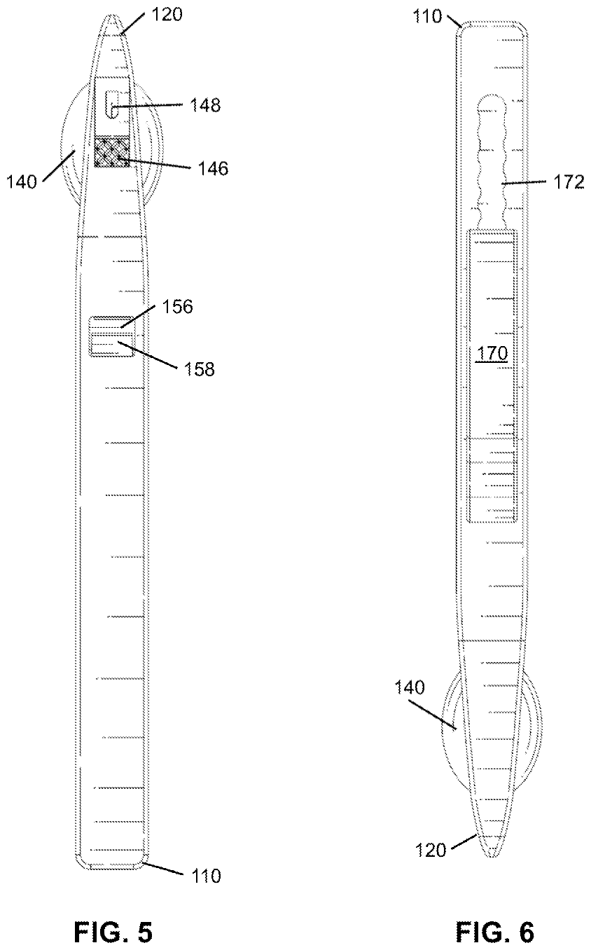

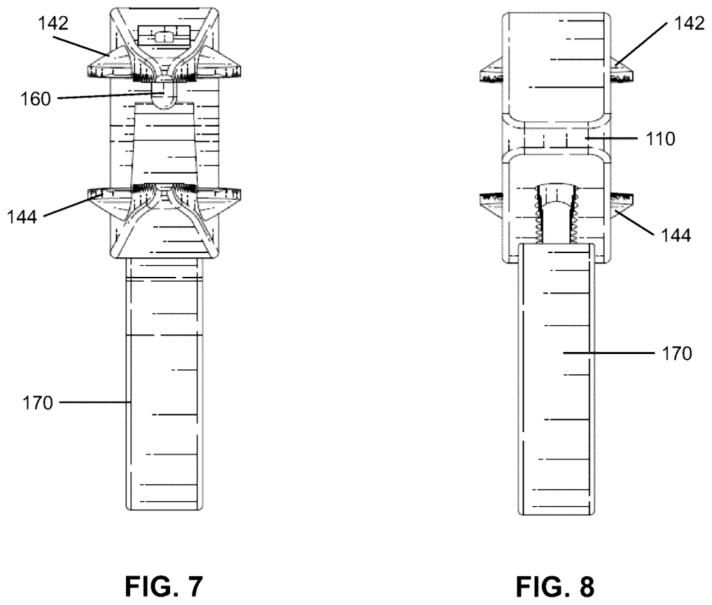

Referring to the drawings in more detail, FIGS. 1-8 show an exemplary embodiment of a suture needle safety device 100 of the present invention. In this exemplary embodiment of the present invention, a suture needle safety device 100 comprises a body having a hinged proximal end 110 opposite a distal, grasping end 120. The suture needle safety device 100 further comprises a first, upper arm 122 and a second, lower arm 124, such arms connected to each other via the proximal, hinged end 110. In this embodiment, the suture needle safety device distal, grasping end 120 comprises distal ends of the first and second arms 122, 124. In an exemplary embodiment, the proximal, hinged end 110 includes an internal torsion spring configured to bias the first and second arms 122, 124 away from each other in an open configuration. In other embodiments, the hinged end 110 includes an alternative biasing feature to bias the device into the open configuration. In further embodiments, the suture needle safety device 100 is constructed from material in a particular shape so as to create an inherent biasing force against closing the first and second arms 122, 124 together.

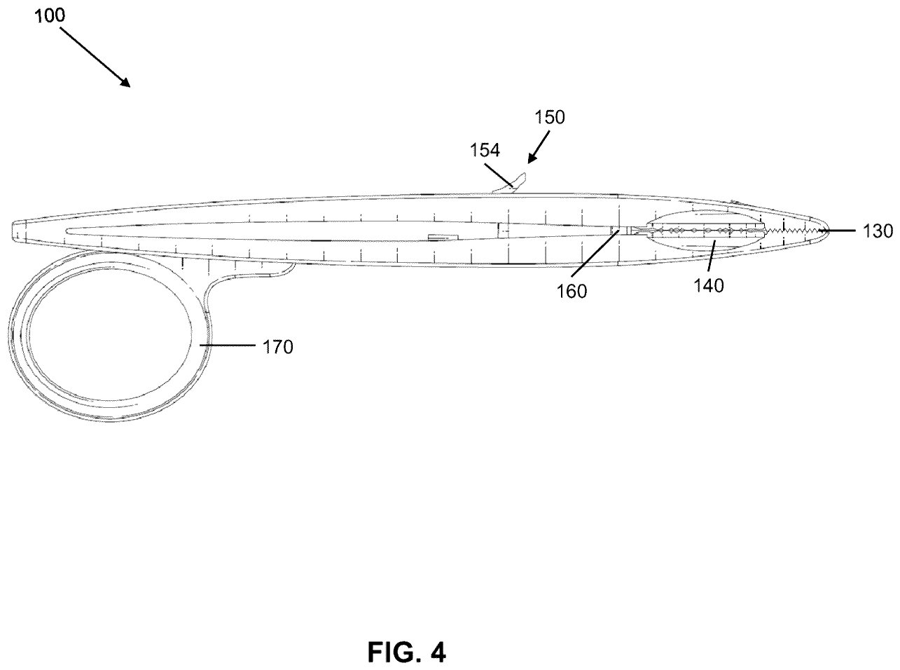

In an exemplary embodiment, a user can apply pressure to each of the first, upper arm 122 and the second, low arm 124 to counteract the hinged end biasing force and to close the arms from the open configuration toward each other toward a closed configuration. FIGS. 1-3 and 5-8 show the suture needle safety device 100 in the open configuration. FIG. 4 shows the suture needle safety device 100 in the closed configuration. In an exemplary embodiment, each of the first arm 122 and the second arm 124 has a slightly concave shape inward, or toward the other arm, similar to the shape of the arms of a standard pair of tweezers. In other embodiments, the first and second arms are substantially straight or any other shape desired for effective handling of a sharp object.

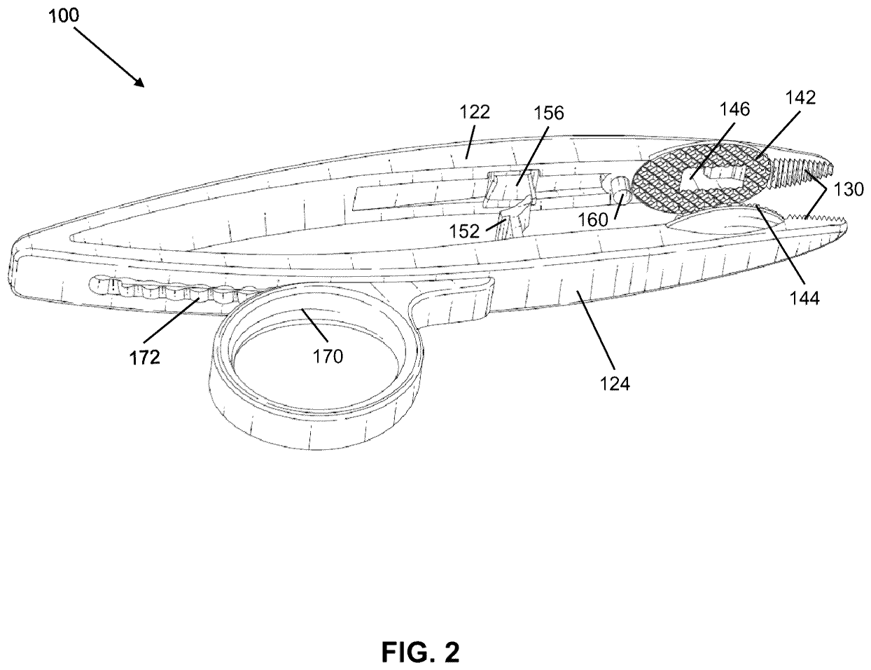

In the embodiment shown in FIGS. 1-8, the suture needle safety device 100 comprises a needle engagement feature 140 positioned near the distal, grasping end 120. In this exemplary embodiment, the needle engagement feature 140 comprises a pair of paddles or substantially oval-shaped needle engagement surfaces 142, 144, one paddle or substantially oval-shaped engagement surface mounted on each of the first arm 122 and the second arm 124 of the needle safety device 100. Such engagement paddles 142, 144 are configured to align and to closely fit together in the closed configuration so as to grasp and retain a suture needle or other sharp object in a fixed position when the device is in the closed configuration. In an exemplary embodiment, the paddles 142, 144 each include a knurled surface for increased gripping ability, as shown in FIGS. 1 and 2. In other embodiments, only one paddle includes a knurled engagement surface. In further embodiments, the paddles include alternative gripping surfaces, such as a grooved surface or mounting one or more gripping material such as but not limited to rubber, polyurethane, or other plastic. In embodiments mounting one or more grip material, such grip material may include ridges or other patterns to further aid gripping of a needle or other object. Other embodiments do not have a grip material or surface, with the closure of the engagement surfaces being sufficient to fixedly retain a needle or other object.

In an exemplary embodiment, the upper arm paddle 142 further defines an upper paddle opening 146 through the paddle 142 and upper arm 122. Such upper paddle opening 146 allows a user to thread a suture strand or other object through the device 100 while retaining a connected object within the engagement feature 140, as desired. Furthermore, in embodiments, the device 100 mounts a suture cutter 148 or alternative blade adjacent to the upper paddle opening 146. Such suture cutter 148 or alternative blade allows a user to cut a suture strand or alternative strand with the present device 100. In embodiments, the suture cutter 148 is angled similar to a floss cutter blade on a standard dental floss container.

In the exemplary embodiment shown in FIGS. 1-8, the suture needle safety device 100 further includes a closing or locking mechanism 150 configured to retain the device 100 in the closed configuration. In this embodiment, the closing mechanism 150 comprises an extension post 152, an extension post receiver 156, and a biased latch 158 positioned within the extension post receiver 156 and configured to engage with the extension post 152. In an exemplary embodiment, the closing mechanism latch 158 is spring loaded. In this embodiment, the second, lower arm 124 of the suture needle safety device 100 mounts the extension post 152 and the first, upper arm 122 defines the extension post receiver 156 with a corresponding latch 158. Nevertheless, in alternative embodiments, the upper arm mounts the extension post and the lower arm mounts the extension post receiver with corresponding latch. In embodiments, the extension post receiver latch can be positioned on any side of the extension post receiver.

In an embodiment, the extension post 152 comprises a post head 154 which extends outward both generally proximally and generally distally from the base of the extension post 152. In an embodiment, the post head 154 is angled with a post head lower side extension facing the side of the post receiver which mounts the latch 158 and a post head higher side extension opposite the lower side extension. Such shape accommodates easy insertion of the extension post 152 in and through the extension post receiver 156 when the device is closed from the open configuration toward the closed configuration. The extension post head 154 is configured to slide against and retract the latch 158 until the post head 154 fully extends through the extension post receiver 156. At such point, the spring-loaded latch 158 expands, contacting and engaging with the extension post 152 below the post head 154. Such engagement of the extension post 152, extension post receiver 156, and corresponding latch 158 lockingly holds the device 100 in a closed configuration until the closing mechanism latch 158 is retracted and the extension post 152 is disengaged from the extension post receiver 156, allowing the hinge end biasing feature to bias the device 100 into the open configuration. In some embodiments, the engagement post 152 is configured to have some generally distal and proximal movement flexibility to aid in retraction of the closing mechanism latch 158 to open the suture needle safety device 100. In other embodiments, a user retracts the latch 158 with the user's finger to disengage the extension post 152 from the extension post receiver 156. In a preferred embodiment, the closing mechanism 150 design allows a user to easily open the latch 158 with one hand to put the device in the open configuration.

In the embodiment shown in FIGS. 1-8, the suture needle safety device distal end further comprises narrowed graspers or pinchers 130 positioned distal of the needle engagement feature 140. Such narrowed graspers 130 allow a user to use the device 100 to grasp other objects, such as but not limited to other surgical procedure materials, such as gauze or other surgical instruments, or tissue surrounding an incision to be sutured shut. In this embodiment, the narrowed graspers 130 are grooved to aid in grasping objects or materials. In other embodiments, the graspers include an alternative gripping surface, such as a knurled surface or mounting one or more gripping material such as but not limited to rubber, polyurethane, or other plastic. In embodiments mounting one or more grip material on the graspers, such grip material may include ridges or other patterns to further aid gripping. Other embodiments do not have a grip material or surface on the graspers.

In the exemplary embodiment shown in FIGS. 1-8, the suture needle safety device further includes a guide piece 160 and corresponding guide receiver 162. In this embodiment, the guide piece 160 is mounted from the first, upper arm 122 and the corresponding guide receiver 162 is positioned on the second, lower arm 124. Nevertheless, in other embodiments, the guide piece 160 may be mounted from the lower arm and the guide receiver 162 positioned on the upper arm. Such guide piece 160 and receiver 162 are configured to help guide a user's closure of the device first arm 122 and second arm 124 into a closed configuration and to help ensure proper alignment of the needle engagement feature 140 and/or graspers 130 during such closure. In this embodiment, the guide piece 160 is substantially cylindrical with a rounded end to ease guiding the guide piece 160 into the guide receiver 162, which is shaped to receive and closely fit around the guide piece 160. In other embodiments, the guide piece can form any other extended shape with a correspondingly shaped guide receiver.

In the embodiment shown in FIGS. 1-8, the suture needle safety device further comprises a finger engagement feature 170 positioned near the proximal end 110 of the device 100. The finger engagement feature 170 is configured for engagement with a user's finger and/or hand to aid the user in holding the device. In an exemplary embodiment, the finger engagement feature 170 is configured to aid in stabilization of the device while using. In an exemplary embodiment, the finger engagement feature 170 of the present invention is configured to aid the user for operation of the device with one hand.

In an embodiment, the finger engagement feature 170 comprises a finger loop. In other embodiments, the finger engagement feature comprises alternative shapes, including but not limited to square; rectangular; oval-shaped; an open (non-enclosed) arc-shape; or a multiple finger, ergonomic shape. In an exemplary embodiment, the finger engagement feature 170 is adjustable to accommodate different user finger or hand sizes and for comfortable fit for a user's finger or hand. In the embodiment shown in FIGS. 1-8, the finger engagement loop 170 is connected to the lower arm 124 of the device 100 via a ratcheted adjustable connection 172 configured to allow slidable ratcheted size and positioning adjustments. In some embodiments, the device includes multiple finger engagement loop sizes. In other embodiments, the finger engagement feature attaches to the rest of the device via threaded connection, snap in connection, compression fitting, articulated attachment, or any other type of attachment. In further embodiments, the finger engagement feature includes adjustable connection to adjust for different user finger or hand sizes by overlapping hook and loop fasteners or buckle systems. In further embodiments, the finger engagement feature is not adjustable and is molded as part of the device or welded to the rest of the device.

In an exemplary embodiment of use of the present invention, during or after a procedure in which a wound or surgical incision is sewn shut using sutures and a suture needle, the surgeon can pick up the suture needle safety device 100 of the present invention with one hand, placing one or more finger through or in contact with the finger engagement feature 170. The user can then place the suture needle on the lower engagement paddle 144 and close the first and second arms 122, 124 of the device 100 with the engagement paddles 142, 144 fixedly retaining at least the sharpened blade of the suture needle. The device closing mechanism 150 will lock the device 100 with the needle fixedly retained within the needle engagement feature 140 until the closing mechanism latch 158 is selectively retracted to open the device 100 into the open configuration. Such device 100 helps to protect doctors, patients, and other medical personnel from puncture and/or contamination when a suture needle is being handled during or after a surgical procedure. As desired, a suture strand can be threaded through the upper paddle opening 146 and/or cut with the suture cutter 148 mounted on the device.

In an exemplary embodiment of the present invention, the surgical needle safety device is made of all or substantially all metal. In an exemplary embodiment, the present device is made of stainless steel. In embodiments, the present device is sterilizable and configured for multiple uses. In other embodiments, the present device is made of silicone, rubber, plastic(s), filament(s), or other puncture resistant materials. In embodiments, the present device is disposable. In embodiments, the device arms include rubber plates or backing to help a user grip the device.

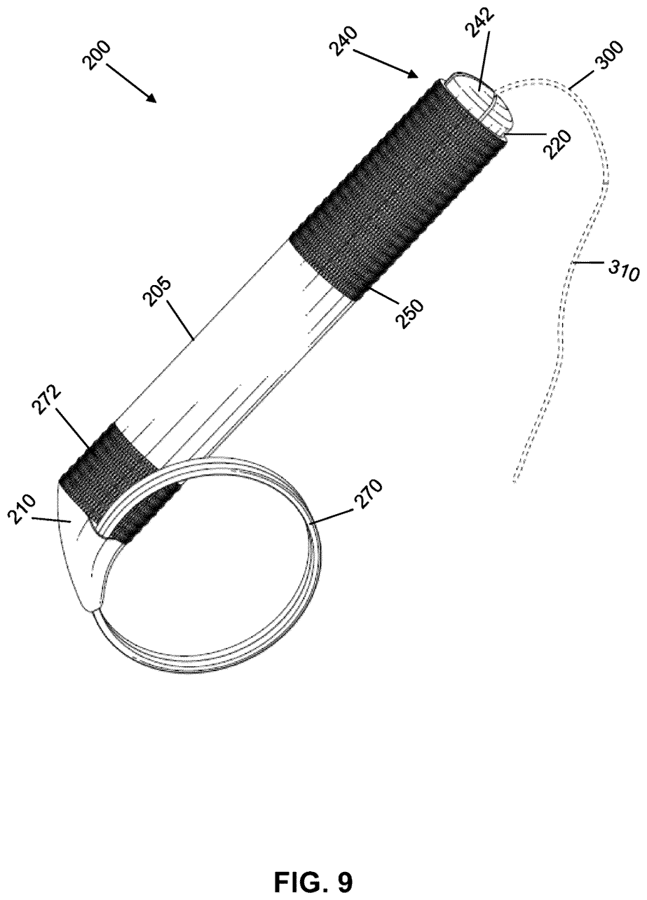

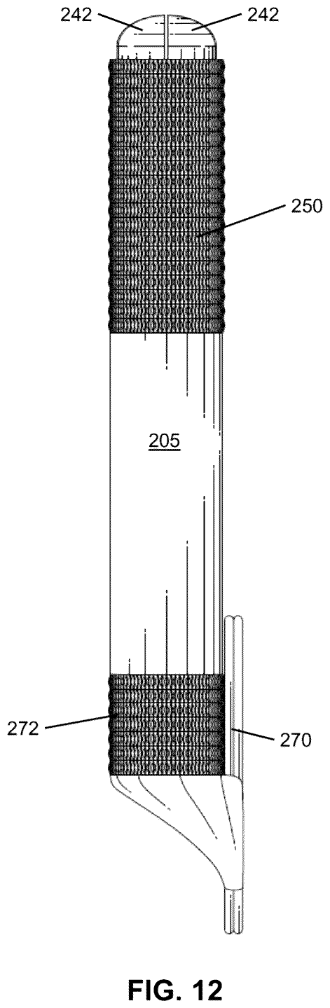

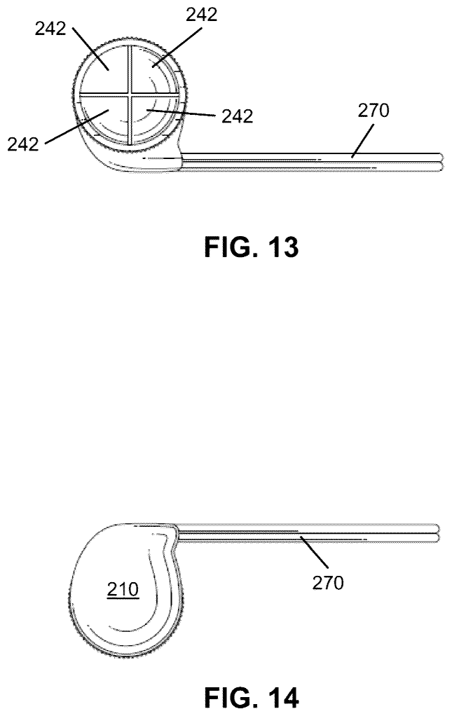

In an alternative embodiment, as shown in FIGS. 9-14, a surgical needle safety device 200 comprises a main body 205 extending between a distal end 220 and a proximal end 210. In an embodiment, the device closing mechanism comprises a spring-loaded slider mechanism 250 mounted on the main body 205, the spring configured to bias the slider mechanism 250 toward the distal end 220 of the main body 205 in an expanded position. In this embodiment, the slider mechanism 250 accommodates a user pulling the slider mechanism 250 proximally, which compresses the spring. In an exemplary embodiment, the slider mechanism 250 is concentric with the device main body 205. In other embodiments, the slider mechanism is located in one plane along the circumferential outer surface of the main body parallel to the longitudinal axis of the main body and slidable in proximal and distal directions. In further embodiments, the slider mechanism is positioned on one side of the main body.

In an exemplary embodiment of the present invention, the slider mechanism 250 includes a knurled outer surface to provide better grip for a user's fingers. In alternative embodiments, the slider mechanism outer surface includes a grip material, such as but not limited to rubber, polyurethane, other plastic, or leather. Other embodiments do not have a grip material or surface.

In this embodiment, the needle safety device 200 needle engagement feature comprises a grasping mechanism 240 at the main body distal end 220 configured to open when the slider mechanism 250 is retracted, or pulled in a proximal direction, and configured to close when the slider mechanism 250 is released and biased distally. In an exemplary embodiment, the grasping mechanism 240 of the present invention opens into a needle receiver cavity 244.

In an embodiment, the grasping mechanism 240 comprises a series of pivotable pieces 242 about a center point or longitudinal axis of the main body 205. In an embodiment, the pivotable pieces 242 are configured for engaging with one another with the device 200 in the closed configuration. In an embodiment, the pivotable pieces 242 define the receiver cavity 244 and are configured to pivot creating an opening into the needle receiver cavity 244 when the device 200 is in the open configuration.

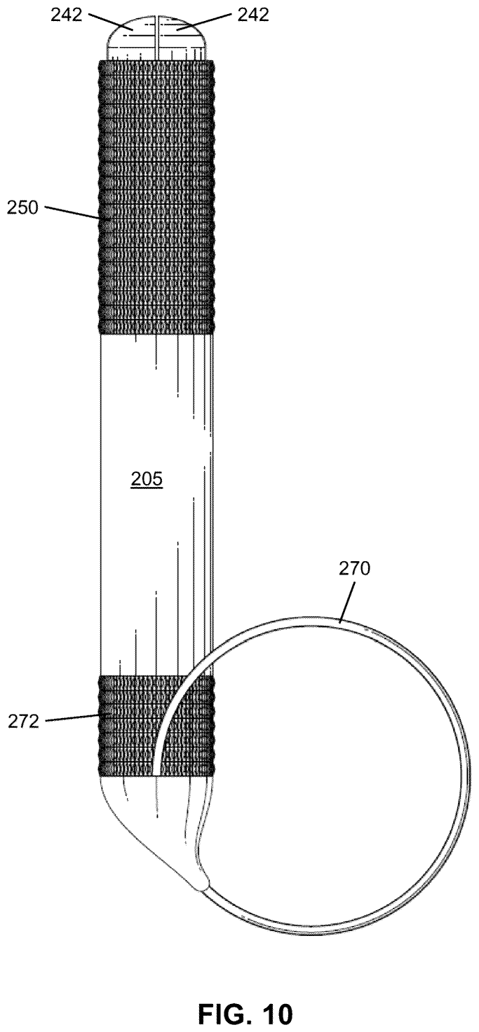

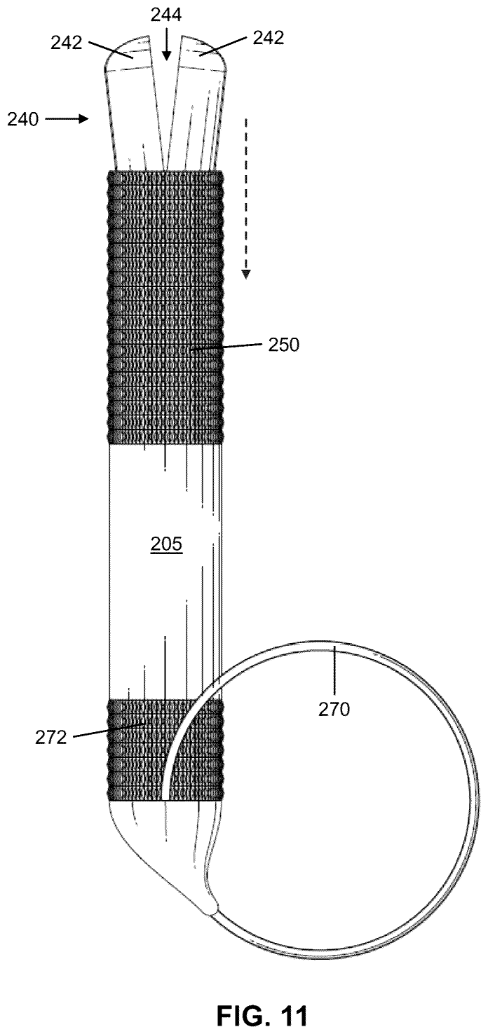

FIG. 9 shows this embodiment of the safety device 200 in a closed configuration with a sharpened portion of a suture needle 300 attached to a suture strand 310 retained within the needle grasping mechanism 240. FIGS. 10 and 12-14 show various views of the device 200 in the closed configuration. FIG. 11 shows the device 200 in an open configuration, with a dashed arrow to illustrate sliding of the slider mechanism 250.

In an embodiment, the grasping mechanism pivotable pieces 242 are shaped such that the slider mechanism 250 biases the pivotable pieces 242 closed when the slider mechanism 250 is biased toward the distal end 220 of the device 200. In an exemplary embodiment, the pivotable pieces 242 are shaped such that the pivotable pieces 242 pivot open when the slider biasing mechanism 250 (e.g., spring) is counteracted by force applied by a user sliding the slider mechanism 250 in a proximal direction. In an embodiment, each pivotable piece 242 comprises a curved, beak-like shape. In embodiments, the pivotable pieces collectively form an ellipsoid or substantially an ellipsoid-shape when engaged with one another. In alternative embodiments, the pivotable pieces comprise other individual and collective shapes. In embodiments, the grasping mechanism comprises two, three, four, five, six, seven, eight, or any other number of pivotable pieces.

In embodiments, the grasping mechanism 240 of the present invention is configured to open because of gravitational forces when the sliding mechanism 250 is retracted. In other embodiments, the grasping mechanism 240 includes a secondary biasing mechanism configured to bias the grasping mechanism pivotable pieces 242 open when the primary biasing mechanism 250 is counteracted by a user. In further embodiments, the grasping mechanism 240 is shaped such that the sliding mechanism 250 of the present invention provides a force when retracted against one or more levers to open the grasping mechanism pivotable pieces 242.

In this embodiment of the present invention, the surgical needle safety device 200 further includes at least one finger engagement feature 270. Such finger engagement feature 270 is configured for engagement with a user's finger and/or hand to aid the user in holding the device 200. In an exemplary embodiment, the finger engagement feature 270 is configured to aid in stabilization of the device 200 while counteracting the biasing force. In an exemplary embodiment, the finger engagement feature 270 of the present invention is configured to aid the user for operation of the device 200 with one hand.

In embodiments, the finger engagement feature 270 comprises a loop or ring shape affixed to the device main body. In other embodiments, the finger engagement feature comprises alternative shapes, including but not limited to square; rectangular; oval-shaped; an open (non-enclosed) arc-shape; or a multiple finger, ergonomic shape.

In embodiments, the present surgical needle safety device includes a variable finger engagement feature. In an embodiment, the finger engagement feature is detachable from the device main body and replaceable with a different size of finger engagement feature to accommodate different user finger or hand sizes (e.g., different ring or loop sizes). In an embodiment, the finger engagement feature attaches to the device main body via threaded attachment. In other embodiments, the finger engagement feature attached to the main body via snap in connection, compression fitting, articulated attachment, or any other type of attachment. In a further embodiment, the finger engagement feature includes adjustable connection to adjust for different user finger or hand sizes, such as but not limited to a slidable ratcheted adjustment, overlapping hook and loop fasteners, or buckle systems. In additional embodiments, the finger engagement feature is molded as part of the main body or welded to the main body.

In embodiments, the suture needle safety device 200 additional includes additional gripping surface 272 near the proximal end 210 near the finger engagement feature 270. In embodiments, the additional gripping surface 272 comprises a knurled surface, a grooved surface, or a grip material, such as but not limited to rubber, polyurethane, other plastic, or leather. In other embodiments, the device does not include additional gripping surface.

In an exemplary embodiment of use of the present invention, during or after a procedure in which a wound or surgical incision is sewn shut using sutures and a suture needle, the surgeon can pick up the suture needle safety device of the present invention with one hand, placing one or more finger through or in contact with the finger engagement feature. The user can then pull back the slider mechanism with the same hand, counteracting the biasing force and opening the gripping mechanism to create an opening into the receiver cavity. With the receiver cavity open, the surgeon can place at least the sharpened portion of the suture needle into the receiver cavity using the surgeon's other hand, and the surgeon can release the slider mechanism, which biases the gripping mechanism to close around the suture needle with the sharpened portion of the needle within the receiver cavity. The suture needle will remain with the sharpened portion within the receiver cavity until someone retracts the slider mechanism again, helping to protect doctors, patients, and other medical personnel from puncture and/or contamination when a suture needle is being handled.

In an exemplary embodiment of the present invention, the surgical needle safety device is made of all or substantially all of metal. In an exemplary embodiment, the present device is made of stainless steel. In embodiments, the present device is sterilizable and configured for multiple uses. In other embodiments, the present device is made of silicone, rubber, plastic(s), filament(s), or other puncture resistant materials. In embodiments, the present device is disposable. In embodiments, the receiver cavity includes rubber plates or backing to help prevent movement of a needle within the cavity and/or to aid in gripping the needle.

In embodiments of the present invention, the biasing mechanism comprises a spring-loaded hinge with a clasp for counteracting the biasing force. In such embodiments, applying force to the clasp opens a two-piece gripping mechanism to provide access to the receiver cavity. In some embodiments, a two-piece gripping mechanism forms a substantial ellipsoid shape to accommodate the curvature of suture needles. In other embodiments, a two-piece gripping mechanism forms any other shape.

In embodiments, the concepts of the present invention are alternatively used for handling other types of needles, various other sharp objects, other contaminated or fouled objects, dirty or messy objects a user does not want to touch, and/or any other objects in a sterile environment.

Certain terminology is used in the description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology shall include the words specifically mentioned, derivatives thereof, and words of similar meaning.

As used in this specification and the appended claims, the singular forms “a”, “an”, and “the” include plural references unless the context clearly dictates otherwise. Thus, for example, a reference to “a method” includes one or more methods, elements, and/or steps of the type described herein and/or which will become apparent to those persons skilled in the art upon reading this disclosure and so forth.

As used in this specification and the appended claims, the use of the term “about” means a range of values including and within 15% above and below the named value, except for nominal temperature. For example, the phrase “about 3 mM” means within 15% of 3 mM, or 2.55-3.45, inclusive. Likewise, the phrase “about 3 millimeters (mm)” means 2.55 mm-3.45 mm, inclusive. When temperature is used to denote change, the term “about” means a range of values including and within 15% above and below the named value. For example, “about 5° C.,” when used to denote a change such as in “a thermal resolution of better than 5° C. across 3 mm,” means within 15% of 5° C., or 4.25° C.-5.75° C. When referring to nominal temperature, such as “about −50° C. to about +50° C.,” the term “about” means ±5° C. Thus, for example, the phrase “about 37° C.” means 32° C.-42° C.

Unless defined otherwise, all technical and scientific terms used herein have the same meaning as commonly understood by one of ordinary skill in the art to which this invention belongs. Although any systems, elements, methods and materials similar or equivalent to those described herein can be used in the practice or testing of the present invention, the preferred systems, elements, and methods and materials are now described. All publications mentioned herein are incorporated herein by reference to describe in their entirety.

“Substantially” means to be more-or-less conforming to the particular dimension, range, shape, concept, or other aspect modified by the term, such that a feature or component need not conform exactly. For example, a “substantially cylindrical” object means that the object resembles a cylinder but may have one or more deviations from a true cylinder. “Comprising,” “including,” and “having” (and conjugations thereof) are used interchangeably to mean including but not necessarily limited to, and are open-ended terms not intended to exclude additional, unrecited elements or method steps.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

It is also to be understood that the following claims are intended to cover all of the generic and specific features of the invention herein described, and all statements of the scope of the invention which, as a matter of language, might be said to fall therebetween.

Having thus described the invention, what is claimed as new and desired to be secured by Letters Patent is:

1. A needle safety device comprising:
a body comprising a proximal end opposite a distal, grasping end;
a needle engagement feature positioned near said distal, grasping end;
wherein said device comprises an open configuration and a closed configuration;
wherein said needle engagement feature is configured to receive a sharp portion of a needle in said open configuration and configured to fixedly retain said sharp portion of said needle in said closed configuration; and
a selectively openable closing mechanism configured to hold said device in said closed configuration.

2. The needle safety device of claim 1, further comprising:
a finger engagement feature connected to said body near said proximal end.

3. The needle safety device of claim 2, wherein:
said finger engagement feature comprises a finger loop; and
said finger loop is positionally adjustable in relation to said body.

4. The needle safety device of claim 1, wherein:
said proximal end comprises a hinged end;
said body further comprising a first arm connected to a second arm by said hinged end; and
said distal, grasping end comprises a distal end of each of said first arm and said second arm.

5. The needle safety device of claim 4, wherein:
said needle engagement feature comprises a first engagement paddle surface positioned on said first arm near said first arm distal end and a second engagement paddle surface positioned on said second arm near said second arm distal end; and
said first engagement paddle surface is configured to align and closely fit together with said second engagement paddle surface in said closed configuration such that said sharp portion of said needle can selectively be fixedly retained between said first engagement paddle surface and said second engagement paddle surface.

6. The needle safety device of claim 5, wherein:
each of said first engagement paddle surface and said second engagement paddle surface comprises a knurled surface.

7. The needle safety device of claim 5, wherein:
said first engagement paddle defines a first paddle opening through said first engagement paddle and said first arm.

8. The needle safety device of claim 7, further comprising:
a suture cutter mounted on said first arm near said first paddle opening.

9. The needle safety device of claim 4, wherein:
said hinged end further comprises an internal biasing feature configured to bias said first arm and said second arm in said open configuration.

10. The needle safety device of claim 4, wherein:
said closing mechanism comprises an extension post mounted on said second arm configured to extension post receiver positioned on said first arm; and
said extension post receiver comprises a selectively retractable, spring-loaded latch configured to engage with said extension post in locking relation in said closed configuration.

11. The needle safety device of claim 10, wherein:
said extension post further comprises a post head shaped to retract said spring-loaded latch when said device is moved from said open configuration to said closed configuration until said post head is fully inserted through said extension post receiver; and
said post head is configured to engage with said extension post in locking relation in said closed configuration.

12. The needle safety device of claim 4, wherein:
each of said first arm and said second arm further comprises a narrowed grasper distal of said needle engagement feature.

13. The needle safety device of claim 4, further comprising:
a guide piece positioned on said first arm; and
a guide receiver positioned on said second arm and configured to receive said guide piece in said closed configuration.

14. The needle safety device of claim 1, wherein:
said needle engagement feature comprises a series of pivotable pieces configured to form a needle receiver cavity therebetween;
said closing mechanism comprising a slider mechanism mounted to said body and biased distally toward said series of pivotable pieces such that said pivotable pieces close together to close said needle receiver cavity; and
said pivotable pieces are configured to pivot to open said needle receiver cavity in said open configuration when a user slides said slider mechanism proximally.

15. The needle safety device of claim 14, wherein:
said needle receiver cavity is configured to selectively receive said sharp portion of said needle in said open configuration and configured to selectively fixedly retain said sharp portion of said needle in said closed configuration.

16. A needle safety device comprising:
a body comprising a proximal, hinged end and a distal, grasping end;
said body further comprising a first arm connected to a second arm by said proximal, hinged end;
wherein said distal, grasping end comprises a distal end of each of said first arm and said second arm;
a needle engagement feature positioned near said distal, grasping end;
wherein said device comprises an open configuration and a closed configuration;
wherein said needle engagement feature is configured to receive a sharp portion of a needle in said open configuration and configured to fixedly retain said sharp portion of said needle in said closed configuration;
said needle engagement feature comprising a first engagement paddle surface positioned on said first arm near said first arm distal end and a second engagement paddle surface positioned on said second arm near said second arm distal end;
wherein said first engagement paddle surface is configured to align and closely fit together with said second engagement paddle surface in said closed configuration such that said sharp portion of said needle can selectively be fixedly retained between said first engagement paddle surface and said second engagement paddle surface;
a selectively openable closing mechanism configured to hold said device in said closed configuration;
said closing mechanism comprising an extension post mounted on said second arm configured to extension post receiver positioned on said first arm; and
said extension post receiver comprising a selectively retractable, spring-loaded latch configured to engage with said extension post in locking relation in said closed configuration.

17. A method of handling a needle, the method comprising the steps of:
providing a needle safety device comprising a body comprising a proximal, hinged end and a distal, grasping end; said body further comprising a first arm connected to a second arm by said proximal, hinged end; wherein said distal, grasping end comprises a distal end of each of said first arm and said second arm; a needle engagement feature positioned near said distal, grasping end; wherein said device comprises an open configuration and a closed configuration; said needle engagement feature comprising a first engagement paddle surface positioned on said first arm near said first arm distal end and a second engagement paddle surface positioned on said second arm near said second arm distal end; wherein said first engagement paddle surface is configured to align and closely fit together with said second engagement paddle surface in said closed configuration; and a selectively openable closing mechanism configured to hold said device in said closed configuration;
placing a sharp portion of a needle on said second engagement paddle surface;
moving said first arm and said second arm toward each other to move said device from said open configuration to said closed configuration;
said first engagement paddle surface and said second engagement paddle surface fixedly retaining said sharp portion of said needle between said first engagement paddle surface and said second engagement paddle surface; and
said closing mechanism holding said device in said closed configuration.

18. The method of claim 17, further comprising the steps of:
selectively opening said closing mechanism; and
removing said needle from said device.

19. The method of claim 17, further comprising the steps of:
selectively opening said closing mechanism; and
disposing of said needle.

20. The method of claim 17, further comprising the step of:
threading a suture strand connected to said needle through a wound or incision to be sutured shut in a patient with said sharp end of said needle fixedly retained within said device.
